(19) United States
(12) Patent Application Publication
(10) Pub. No.: US 2006/0111756 A1
(43) Pub. Date: May 25, 2006

(54) Method and apparatus for a neuromuscular stimulator

(76) Inventor: Henry H. Chang, West Covina, CA (US)

Correspondence Address: PHILIP K. YU, 20955 PATHFINDER ROAD, SUITE 100, DIAMOND BAR, CA, 91765, US

(21) Appl. No.: 10/997,669

(22) Filed: Nov. 23, 2004

(51) Int. Cl. A61N 1/18

(52) U.S. Cl. 607/48

(57) ABSTRACT

A portable neuromuscular stimulator is disclosed. The neuromuscular stimulator for use with a patient during general anesthesia comprises a housing, a pair of electrode terminals disposed on the outside of the housing, a pulse generator within the housing, wherein the pulse generator is disposed to controllably generate one of TOF and Tetanus stimulations to the electrode terminals, and the pulse generator having an intensity controller to controllably vary the level of intensity of the stimulations. The stimulator also has a liquid crystal display (“LCD”) on the outside of the housing, with the LCD being disposed to display a mode of operation and a level of intensity representative of the stimulation being applied to the electrode terminals from said pulse generator.

### RELATED FIELD

The invention relates to neuromuscular stimulators used by anesthesiologists on patients in surgery, and more particularly relates to the neuromuscular stimulators for measuring the degree of a patient's muscle paralysis after administration of muscle blocking agent during general anesthesia.

### ART BACKGROUND

After a patient has been administered general anesthesia for surgery, the attending anesthesiologist needs to monitor the patient's muscular response to electric stimulations. By observing the patient's response to such electric stimulations, the anesthesiologist can determine how much recovery in muscular power the patient has achieved before the doctors can begin to awaken the patient. Such careful monitoring is crucial since the consequences could be disastrous if the patient is brought back from anesthesia without such monitoring. If the patient had not fully recovered from the general anesthesia, the patient would wake up, but would still remain paralyzed and unable to function normally. Such paralysis would be very dangerous since the patient may not even be able to breathe on his own after being awakened. Without adequate supply of oxygen to the brain, the patient could easily sustain permanent brain damage.

Conventionally, anesthesiologists use a muscle stimulator to measure the degree of muscle paralysis after administration of a muscle blocking agent. This stimulator device typically generates two modes of stimulation: “Twitch” and “Tetanus.” As is well known in the field of anesthesiology, a twitch stimulation typically gives a pulse at about 2 Hz, or at ½ second interval, while the Tetanus stimulation generates a continuous stimulation lasting 5 seconds in duration. Both stimulation modes are commonly used to observe the degree of muscle response of the patient's finger(s) or hand, when they are raised from a resting position to a upright position when stimulated. Additionally, a Train-of-Four (“TOF”) simulation has been introduced to the field in recent years. The use and efficacy of such stimulating modes have been studied by WAUD, B. E., M. D., et al., “The Relationship between the Response to ‘Train-of-Four’ Stimulation and Receptor Occlusion during Competitive Neuromuscular Block,” ANESTHESIOLOGY, vol. 37, No. 4, October 1972, and by KOPMAN, Aaron F., M. D., et al., “Relationship of the Train-of-four Fade Ratio to Clinical Signs and Symptoms of Residual Paralysis in Awake Volunteers,” ANESTHESIOLOGY, vol. 86, No. 4, April 1997. The disclosure of the above two articles is hereby incorporated by reference.

One such conventional stimulator device is a palm-sized handheld unit, about the dimension of a garage door opener. FIG. 1 illustrates a simplified diagram of the muscle stimulator manufactured by Life-Tech, Inc., of Stafford, Tex., Model MS-1B, presumably under the mark “MiniStim.” This kind of a stimulator device has two metal electrodes on top, producing negative and positive charges, and can generate Tetanus pattern at 50 Hz and Twitch pulses at 2 Hz. When in use, the electrodes need to touch the patient's forearm at, or around, the nerve distribution in order to detect the degree of muscle twitching during general anesthesia. To ensure contact, the bulky device has to be held by the anesthesiologist, with the two electrodes being hard-pressed against the patient's skin for delivering the stimulation.

While this device generates a stimulation pattern of pre-set intensity, the degree of electricity transmitted through the skin tends to be uneven, due to the uneven pressure applied to the skin and the moisture content of the skin. As such, the interpretation of the result can be somewhat inaccurate and inconsistent, due to these variables. Since it only delivers either Tetanus or Twitch simulations, its results do not provide as accurate a picture of the patient's recovery as the TOF's results. Also, it causes discomfort to the patient, when the metal electrodes are hard-pressed against the skin. Such discomfort is exacerbated, when the device is often overpowered, in order to provoke muscular response, to a degree that causes pain to the patient. Finally, since there is no visual display on the device, there is no assurance that a stimulation pattern of the same intensity is delivered to the patient, if measurement has to be taken again, after the device has been removed from the patient or powered off.

The other kind of a conventional stimulator device uses two wires of positive and negative charges, which are attached by alligator clips to EKG-like electrode pads on the skin of the patient's forearm or temporal facial area. The degree of muscle twitching of the patient's fingers or facial muscles is then observed. However, the stimulation signals generated by this device cannot be adjusted, neither in intensity level nor in signal pattern. Such limitation means that the stimulations delivered to the patient will cause discomfort to the patient, much in the same way as the aforementioned device. Also, there can be no assurance that the same stimulation is delivered to the patient if measurement has to be taken again, after the device has been removed from the patient or powered off.

Therefore, it would be desirable to have a stimulator device that can provide adjustable stimulation, in both Tetanus and TOF, both in intensity and in signal pattern, to the patient.

It would also be desirable to have a stimulator device that can allow the anesthesiologist to have a visual display and control of the status and mode of the stimulator device when in use.

It would further be desirable to have a device that can provide a convenient and consistent contact with the patient while minimizing discomfort to the patient.

### SUMMARY OF THE INVENTION

A portable neuromuscular stimulator is disclosed. The neuromuscular stimulator for use with a patient during general anesthesia comprises a housing, a pair of electrode terminals disposed on the outside of the housing, a pulse generator within the housing, wherein the pulse generator is disposed to controllably generate one of TOF and Tetanus stimulations to the electrode terminals, and the pulse generator having an intensity controller to controllably vary the level of intensity of the stimulations. The stimulator also has a liquid crystal display (“LCD”) on the outside of the housing, with the LCD being disposed to display a mode of operation and a level of intensity representative of the stimulation being applied to the electrode terminals from said pulse generator.

In another preferred embodiment, the stimulator further comprises a disposable flexible pad, with the pad having on a first surface mating electrode terminals for removably attaching to the pair of electrode terminals of the housing, and on a second surface a pair of gel pads electrically connected to the mating electrode terminals for removably attaching to the patient's skin.

In yet another preferred embodiment, the stimulator further comprises a pair of extension wires for connecting between the stimulator housing and the disposable flexible pad.

### BRIEF DESCRIPTION OF THE DRAWINGS

FIG. 1 illustrates a simplified diagram of a conventional stimulator device with metal electrode terminals.

FIG. 2 illustrates an external design of an exemplary stimulator device 20 with a disposable bi-polar pad 25 in accordance with the present invention.

FIG. 3 illustrates an exemplary functional block diagram of a stimulator device in accordance with the present invention.

FIG. 4 illustrates an exemplary software process flow chart of a stimulator device in accordance with the present invention.

### DETAILED DESCRIPTION OF THE PREFERRED EMBODIMENT

A portable neuromuscular stimulator is disclosed. In the following detailed description, numerous specific details are set forth to provide a full understanding of the present invention. It will be obvious, however, to those ordinarily skilled in the art that the present invention may be practiced without some of these specific details. In other instances, well-known structures and techniques have not been shown in detail so as to avoid unnecessarily obscure the present invention.

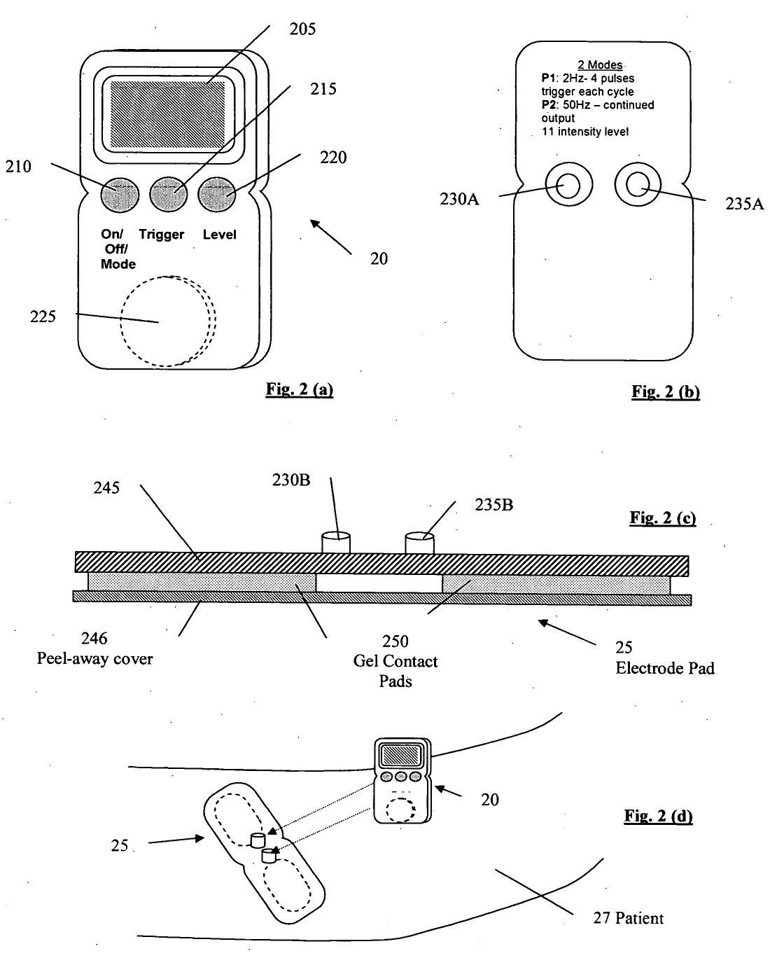

Reference is made to FIGS. 2 (a)-(d), where an external design of an exemplary stimulator device 20 with a disposable bi-polar pad 25 in accordance with the present invention is illustrated. Referring to FIGS. 2 (a)-(d), the stimulator device 20, which may have an exemplary dimension of 53 mm×30 mm×10 mm, has three control buttons: On/Off 210, Trigger 215 and Level 220. A lithium battery, for example, a 3V CR2025 battery, which is placed into a battery receptacle on the side of the device, is used to power this stimulator device 20. To help the anesthesiologist monitor the operation of the device, an LCD display 205 is positioned on the stimulator device. On the back side of the device 20, electrode terminals 230A, 235A, such as the female button-type clip receptacle, are positioned to provide electric contact to a one-piece electrode pad 25.

Upon the user's pressing of the trigger button 215, the device 20 can generate either 4 pulses at 2 Hz (“P1 mode”), or a continuous pulse at 50 Hz (“P2 mode”). Also, the intensity level of the pulse can be adjusted for each mode. For example, as shown in Table 2 (e), for P1, the intensity level may increment through 11 levels (amplitude in mA, peak-to-peak), e.g. 0, 20, 30, 40, 50, 55, 60, 65, 70, 75 and 80. The intensity level for P2 may step through 0, 20, 25, 30, 35, 40, 45, 50, 55, 60 and 65. However, the intensity levels, or the increments, can be easily modified by those skilled in the art when implementing the stimulator device in accordance with the present invention based on their own operating requirements. By having a visual display of the mode and intensity level on the LCD 205, the anesthesiologist can be assured that the same, consistent stimulation be applied to the patient for accurate monitoring, even if the device is powered off or removed from the patient briefly.

Referring to FIGS. 2 (c)-(d), a flexible, single-piece, bi-polar electrode pad 25 is shown in a simplified cross-sectional view. At the top layer of the pad 25, two protruded male electrode terminals 230B, 235B, allow the stimulator device 20 to be removably attached to the electrode pad 25 through its female receptacle 230A, 235A. This kind of male-female, push-button metal clipper is quite common to those skilled in the field. The electrode terminals 230B, 235B, are electrically connected to the gel pads 250 through a plastic insulator layer 245. The gel pads 250 can be adhesively attached to a patient's skin after the peel-away cover 246 is removed. As shown in FIG. 2 (d), the electrode pad 25 can be easily applied to the patient's skin 27 to provide a consistent and complete contact surface, thanks to the flexible shape of the electrode pad 25, which can accommodate the anatomical shape of the patient, e.g. on the forearm or on the temporal facial area. Once the pad 25 is placed on the patient, the stimulator device 20 can be easily attached to the pad 25 by engaging the terminals 230A, 235A on the stimulator device 20 to the terminals 230B, 235B on the electrode pad 25. More importantly, the electrode pad 25 can remain on the patient's skin throughout the procedure, even if the stimulator device 20 may be removed from time to time. All the doctor has to do is simply re-engaging the stimulator device 20 back onto the electrode pad 25. Preferably, the electrode pad 25 is disposable so as to prevent cross contamination to the patients.

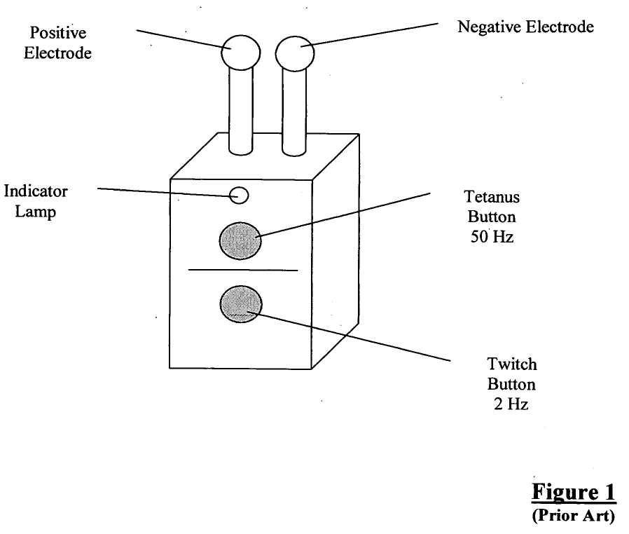

As described in the background of the invention, the conventional technique of applying stimulation is either through direct skin contact by two metal electrodes from the stimulator box (as exemplified in FIG. 1), or through using wires to connect two separate EKG pads on the patient. In contrast, the stimulator device 20 and the disposable flexible electrode pad 25 of the present invention provide a consistent and uniform contact to the patient, through the single bi-polar electrode pad 25. Such contact can be made with or without the use of extension wires between the stimulator device and the patient, although it is preferable to attach the stimulator device 20 directly on the electrode pad 25 itself.

Additionally, multiple levels of intensity and different modes of pulses, i.e. TOF and Tetanus, are provided by the stimulator device 20 of the present invention. The adjustable intensity levels allow the output of the stimulator device 20 to be tested before the patient is under general anesthesia to find the optimal comfort level of stimulation, so that any unnecessary high voltage pulses can be avoided during anesthesia. The level and mode of stimulation displayed on the LCD 205 allow the attending doctors to accurately monitor and record the event for delivering better anesthesia care to the patients.

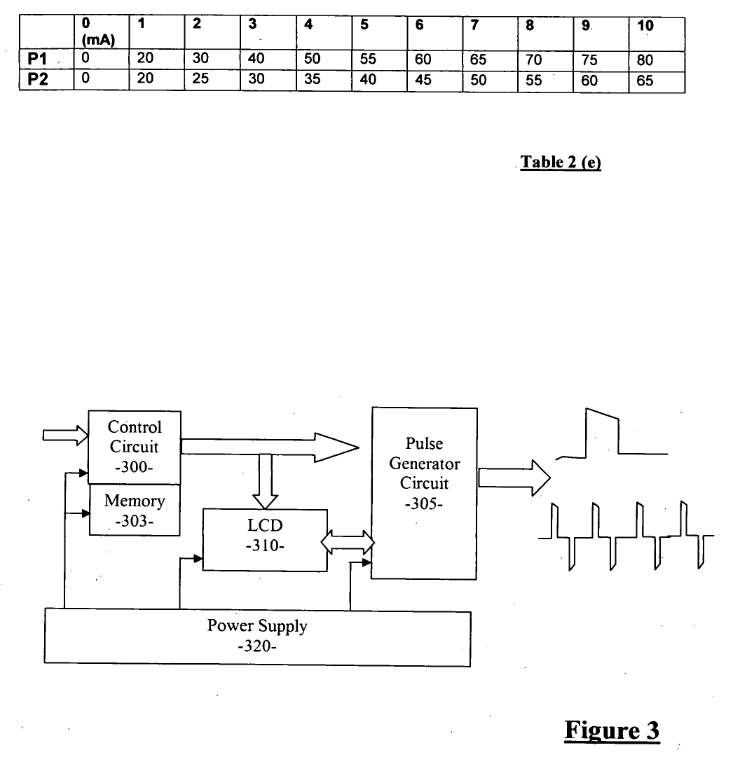

Reference is now to FIG. 3, where an exemplary functional block diagram of the stimulator device in accordance with the present invention is illustrated. A control circuit 300 and memory 303 receive the user's input with respect to the mode and intensity of stimulation and convert it into instructions for the pulse generator 305. The pulse generator circuit 305 then generates either the tetanus pulses or TOF pulses at the specified intensity level. The mode and intensity data are also displayed on the LCD 310 of the stimulator device. The power supply 320, e.g. the 3V lithium battery, provides power to the components on board. It should be noted that a pulse generator circuit is readily available as an integrated circuit chip (“IC”), possibly with the control circuit also implemented on the circuit board. Currently, an 8-bit microcontroller from Holtek Semiconductor, Inc., of Hsinchu, Taiwan, Republic of China, with Part No. HT 49C30, is used to provide the pulses and drive the LCD, based on the user input. Of course, those skilled in the art may find other microcontrollers or microprocessors just as suitable for their application in accordance with the teaching of the present invention.

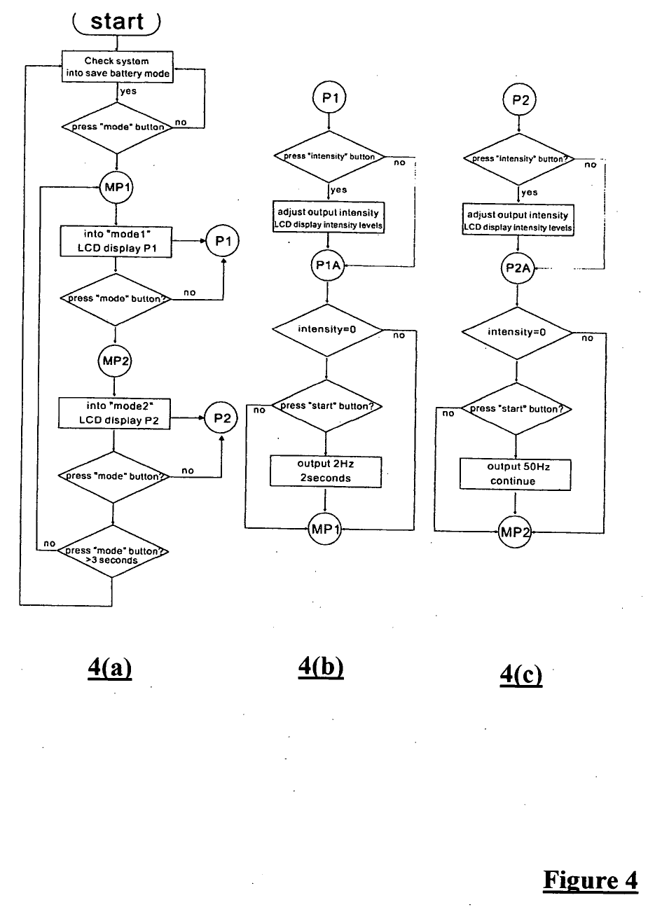

Reference is to FIGS. 4 (a)-(c), where an exemplary process flow of the stimulator device 20 in accordance with the present invention is illustrated. As shown in FIG. 4 (a), from “Start,” the stimulator device continuously checks if the “Mode” button is pressed, so that it can be out of the “battery save” mode. When the “Mode” button is pressed, the “P1” mode is first activated with the LCD displaying “P1” accordingly. It stays in the “P1” mode until the “Mode” button is pressed again. When the “Mode” button is pressed again, the “P2” mode is activated with the LCD displaying “P2” accordingly. If the “Mode” button is pressed for an extended duration, e.g. more than 3 seconds, the stimulator device goes into its “battery save” mode.

FIG. 4 (b) further illustrates the process flow of the stimulator device in P1 mode. When the “Intensity” button is pressed successively, the intensity level can be incremented from Level 0 to Level 10, with the LCD displaying the intensity levels. Then, pressing the “Start” button will activate the 2 Hz TOF output of the stimulator device. If the “Intensity” button is not pressed, then the default Level 0 is activated. As a safety precaution, the stimulator device preferably always starts at the lowest level in order to avoid overpowering the patient. FIG. 4 (c) illustrates the same process flow for the case of 50 Hz Tetanus output in P2 mode.

Although the invention is described herein with reference to the preferred embodiment, one skilled in the art will readily appreciate that other applications may be substituted for those set forth herein without departing from the scope of the present invention. Accordingly, the invention should only be limited by the claims included below.

What is claimed is:

1. A neuromuscular stimulator for use with a patient during general anesthesia, comprising:
a housing;
a pair of electrode terminals disposed on the outside of said housing;
a pulse generator within said housing, said pulse generator being disposed to controllably generate one of TOF and Tetanus stimulations to said electrode terminals, said pulse generator having an intensity controller to controllably vary the level of intensity of said stimulations;
a liquid crystal display (“LCD”) on the outside of said housing, said LCD being disposed to display a mode of operation and a level of intensity representative of the stimulation being applied to said electrode terminals from said pulse generator.

2. The neuromuscular stimulator of claim 1, further comprising:
a disposable flexible pad, said pad having on a first surface mating electrode terminals for removably attaching to said pair of electrode terminals of said housing, and on a second surface a pair of gel pads electrically connected to said mating electrode terminals for removably attaching to said patient's skin.

3. The neuromuscular stimulator of claim 1, further comprising:
a pair of connecting wires, a first end of said connecting wires being disposed to be removably connected to said electrode terminals on said housing;
a disposable flexible pad, said flexible pad having on a first surface mating electrode terminals for removably attaching to a second end of said connecting wires, and on a second surface, a pair of gel pads electrically connected to said mating electrode terminals for removably attaching to said patient's skin.

4. The neuromuscular stimulator of claim 2, further comprising a battery unit for powering said stimulator.

5. The neuromuscular stimulator of claim 3, further comprising a battery unit for powering said stimulator.

6. The neuromuscular stimulator of claim 2, wherein said pair of electrode terminals on said housing is a pair of receptacles, each having a spring clip within for reinforcing contact.

7. The neuromuscular stimulator of claim 3, wherein said pair of electrode terminals on said housing is a pair of receptacles, each having a spring clip within for reinforcing contact.

8. The neuromuscular stimulator of claim 2, wherein said flexible pad comprises a pair of gel pads for adhesively and removably attaching to said patient's skin.

9. The stimulator kit of claim 1, wherein said TOF stimulation comprises a train of 4 pulses at 2 Hz for each trigger.

10. The stimulator kit of claim 1, wherein said Tetanus stimulation comprises a continuous pulse at 50 Hz.

11. A neuromuscular stimulator kit for use with a patient during general anesthesia, comprising:
a portable housing;
a pair of electrode terminals disposed on the outside of said housing;
a pulse generator within said housing, said pulse generator being disposed to controllably generate one of TOF and Tetanus stimulations to said pair of electrode terminals, said pulse generator having an intensity controller to controllably vary the level of intensity of said stimulations;
a liquid crystal display (“LCD”) on the outside of said housing, said LCD being disposed to display a mode of operation and a level of intensity representative of the stimulation being delivered to said pair of electrode terminals from said pulse generator;
a flexible pad, said pad having on a first surface mating electrode terminals for removably attaching to said pair of electrode terminals of said housing, and on a second surface a pair of gel pads electrically connected to said mating electrode terminals for removably attaching to said patient's skin.

12. The stimulator kit of claim 11, wherein said TOF stimulation comprises a train of 4 pulses at 2 Hz for each trigger.

13. The stimulator kit of claim 11, wherein said Tetanus stimulation comprises a continuous pulse at 50 Hz.

14. The stimulator kit of claim 11, wherein said flexible pad is disposable.

15. The stimulator kit of claim 14, further comprising:
a pair of extension wires for removably connecting between said housing and said flexible pad.

16. A method of applying neuromuscular stimulation to a patient during general anesthesia, comprising the steps of:
providing a portable stimulator unit, said stimulator unit being disposed to controllably generate one of TOF and Tetanus stimulations with variable intensity levels, said stimulator unit having a first pair of electrode terminals;
attaching a disposable flexible pad to said patient, said pad having a pair of gel pads for removably attaching to said patient, said pad having a second pair of electrode terminals;
attaching said stimulator unit to said pad by connecting said first and second pairs of electrode terminals together;
selecting one of TOF and Tetanus stimulations of said stimulator unit;
selecting an intensity level of said stimulator unit;
activating said stimulator unit.

17. The method of claim 16, wherein said step of attaching said stimulator unit to said pad further comprises attaching a pair of connecting wires between said stimulator unit and said pad.

18. The method of claim 16, wherein said stimulator unit comprises:
a housing;
a pair of electrode terminals disposed on the outside of said housing;
a pulse generator within said housing, said pulse generator being disposed to controllably generate one of TOF and Tetanus stimulations to said electrode terminals, said pulse generator having an intensity controller to controllably vary the level of intensity of said stimulations;
a liquid crystal display (“LCD”) on the outside of said housing, said LCD being disposed to display a mode of operation and a level of intensity representative of the stimulation being applied to said electrode terminals from said pulse generator.
